(19) United States
(12) Patent Application Publication
(10) Pub. No.: US 2005/0129754 A1
(43) Pub. Date: Jun. 16, 2005

(54) Injectable anesthetic formulation

(76) Inventors: Elspeth J. Gray, Kent (GB); Rodney L. Horder, Libertyville, IL (US); Brian C. Withers, Kent (GB)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/035,935

(22) Filed: Jan. 14, 2005

(30) Foreign Application Priority Data
May 27, 1999 (GB) 9912446.3

(51) Int. Cl. A61K 9/127; A61K 31/02

(52) U.S. Cl. 424/450; 514/743

(57) ABSTRACT

An injectable anesthetic formulation. The formulation contains a halogenated anesthetic in an amount not greater than approximately 24% v/v of the formulation and an emulsification adjuvant in an amount from approximately 8% to approximately 32% v/v of the formulation. In addition, the formulation contains lecithin in an amount from approximately 1.2% to approximately 2.4% w/v of the formulation and a co-emulsifier in an amount not greater than approximately 1% w/v of the formulation.

### TECHNICAL FIELD OF THE INVENTION

The field of the present invention is anesthetics. More particularly, this invention pertains to an injectable anesthetic formulation.

### BACKGROUND OF THE INVENTION

Inhalation anesthetics such as isoflurane and sevoflurane are commonly used for anesthetizing patients for medical procedures. Although inhalation anesthetics are suitable for many medical procedures, they do have certain disadvantages. For example, induction of anesthesia by inhalation can be relatively slow in some patients. Further, the use of inhalation anesthetics requires the patient to breathe the anesthetic using a gas containment mask. The wearing of such a containment mask can be upsetting for some patients, particularly children. For these and other reasons, rapid anesthetic induction is commonly performed by intravenous injection using relatively short acting agents such as propofol. Inhalation anesthetics are then used to maintain the anesthetized condition.

Inhalation anesthetics such as isoflurane and sevoflurane generally have been deemed unsuitable for parenteral administration due to their low aqueous solubility, thereby making it difficult to formulate them for intravenous administration, i.e., their low solubility results in unacceptably large dose volumes.

The need for a suitable formulation for injection has been recognized in the art. For example, U.S. Pat. No. 5,637,625 discloses a phospholipid-coated, microdroplet propofol formulation. The disclosed formulation is devoid of fats and triglycerides so that the formulation provides sedation without fat overload. In addition, the formulation is free of nutrients capable of supporting bacterial growth, thereby providing the formulations with an increased shelf life. In addition, the use of lecithin-coated microdroplets of methoxyflurane was described in “Pharmacokinetics of Methoxyflurane After its Intra-dermal Injection as Lecithin-coated Microdroplets,” published in J. Controlled Release (1989), 9(1), 1-12. However, neither of these disclosures suggests the possibility of using an emulsion formulation of an inhalation anesthetic.

### SUMMARY OF THE INVENTION

The present invention is directed to an injectable anesthetic formulation. In a first embodiment of the present invention, the formulation contains a halogenated anesthetic in an amount not greater than approximately 24% v/v of the formulation and an emulsification adjuvant in an amount from approximately 8% to approximately 32% v/v of the formulation. The formulation further contains lecithin in an amount from approximately 1.2% to approximately 2.4% w/v of the formulation and a co-emulsifier in an amount not greater than approximately 1% w/v of the formulation.

### DETAILED DESCRIPTION OF THE INVENTION

Although the present invention has been described herein in connection with certain exemplary and preferred embodiments, it will be appreciated by one of ordinary skill that various modifications can be made to the invention without departing from the scope of the invention, such scope being defined by the appended claims.

The anesthetic formulations of the present invention have use in inducing maintaining anesthesia in humans and animals. The formulations include a halogenated volatile anesthetic having a boiling point between approximately 20° and approximately 60° C. Such halogenated volatile anesthetics include, but are not necessarily limited to, desflurane, isoflurane, enflurane, halothane, and sevoflurane. Each of these anesthetics is well-known in the art. Although the examples set forth herein disclose formulations containing isoflurane, it is to be understood that any halogenated anesthetic having the desired boiling point can be used in the formulations of the present invention.

The formulations of the present invention further include an emulsification adjuvant such as soybean oil. Those of ordinary skill in the art will appreciate that other emulsification adjuvants having the characteristics of soybean oil can be used without departing from the spirit and scope of the present invention.

It has been found that a minimum ratio of 1 part emulsification adjuvant to 3 parts anesthetic is required in order to provide a stable emulsion. Further, it has been found preferable to provide an anesthetic formulation in which the total volume of dispersed phase, i.e., anesthetic and emulsification adjuvant, in the anesthetic formulation is below approximately 32% v/v in order to ensure that the viscosity of the resulting anesthetic formulation is acceptable for injection. In a preferred embodiment of the anesthetic formulation of the present invention, the halogenated anesthetic is present in an amount not greater than approximately 24% v/v while the emulsification adjuvant is present in an amount between approximately 8% and approximately 32% v/v.

The anesthetic formulation of the present invention further includes an emulsifier such as lecithin. Those of ordinary skill in the art will appreciate that other emulsifiers having the characteristics of lecithin can be used without departing from the spirit and scope of the present invention. The emulsifier is preferably present in an amount between approximately 0.6% and approximately 2.4% w/v. It has been found that emulsifier levels between approximately 1.2% and approximately 2.4% w/v are more preferable, and that emulsifier levels between approximately 1.8% and approximately 2.4% are most preferable in connection with the anesthetic formulation of the present invention.

The anesthetic formulation of the present invention further includes a co-emulsifier. An example of a co-emulsifier useful in connection with the anesthetic formulation of the present invention is a polyoxypropylene/polyoxyethylene block copolymer having a formula HO(C₂H₄O)_{b}(C₃H₆O)ₐ(C₂H₄O)_{b}H where a is an integer such that a molecular weight represented by a polyoxypropylene portion of the copolymer is between approximately 900 to 15000, and b is an integer such that a molecular weight represented by a polyoxyethylene portion of the copolymer constitutes between approximately 5% and 90% of the copolymer. Those of ordinary skill in the art will appreciate that other co-emulsifiers having the characteristics of a polyoxypropylene/polyoxyethylene block copolymer can be used without departing from the spirit and scope of the present invention. In one embodiment of the present invention, poloxamer 188 is used as a co-emulsifier. The co-emulsifier is preferably present in an amount not greater than approximately 1% w/v, as explained in greater detail below, and more preferably in an amount not greater than approximately 0.96%.

It has been discovered that at a total emulsifier lever (i.e., emulsifier content plus co-emulsifier content) of 1.8% w/v, a ratio of 8 parts of lecithin to 2 parts of poloxamer 188 provided desirable results in a 20% v/v isoflurane formulation due to an apparent reduction in droplet size and an increased resistance to creaming. Creaming is a form of emulsion instability well known in the art. However, it has been discovered that no stability benefits are obtained by the inclusion of poloxamer 188 in a 10% v/v isoflurane formulation. Accordingly, certain formulations in accordance with the present invention need not include a co-emulsifier such as poloxamer 188. Accordingly, as used herein, the term “in an amount not greater than” is intended to include the complete absence of a co-emulsifier from the anesthetic formulation of the present invention.

The anesthetic formulation of the present invention may further include a tonicifier such as glycerol. The tonicifier is used to adjust the tonicity of the anesthetic formulation to the tonicity of the patient's blood plasma. In a preferred embodiment of the anesthetic formulation of the present invention, the tonicifier is present in an amount between approximately 1% and approximately 4% of the formulation.

The anesthetic formulation of the present invention may also include a pH adjustor in an amount sufficient to adjust the pH of the formulation to between approximately 6 and approximately 9, thereby making it suitable for injection and also for optimizing the stability of the emulsifier. A variety of known pH adjustors such as sodium hydroxide can be used in connection with the formulation of the present invention.

The preferred anesthetic formulation of the present invention further includes a vehicle for injection in a quantity sufficient for injection of the anesthetic formulation. Water can be used as the vehicle for injection.

The following examples are provided for the purpose of providing a further understanding of the anesthetic formulations of the present invention and are not intended to be limiting of the invention claimed in the appended claims.

### EXAMPLE 1

A 10% v/v formulation of isoflurane was prepared. Each 100 ml of the resulting anesthetic formulation contained:

| | |
|---|---|
| isoflurane | 10 ml |
| soybean oil | 10 ml |
| glycerol | 2.5 g |
| lecithin | 1.8 g |
| distilled water | q.s. |

The soybean oil used in this example was winterized.

### EXAMPLE 2

A 20% v/v formulation of isoflurane was prepared. Each 100 ml of the resulting anesthetic formulation contained:

| | |
|---|---|
| isoflurane | 20 ml |
| soybean oil | 10 ml |
| glycerol | 2.5 g |
| lecithin | 1.6 mg |
| poloxamer 188 | 0.18 g |
| distilled water | q.s. |

The soybean oil used in this example also was winterized. A pH adjustor (0.1 M sodium hydroxide) was added to adjust the pH of the resulting anesthetic formulation to between approximately 8 and approximately 9 prior to autoclaving of the resulting formulation.

Anesthetic formulations prepared in accordance with the present invention are suitable for terminal sterilization by autoclaving, e.g., heating to a temperature of approximately 121° C. for approximately 15 minutes. This characteristic of the anesthetic formulations of the present inventions obviates the need for sterile processing.

Although the present invention has been described herein in conjunction with certain preferred embodiments and examples, it will be appreciated that certain modifications to the anesthetic formulation of the present invention can be made without departing from the intended spirit and scope of the present invention which is defined by the appended claims.

1. An injectable anesthetic formulation comprising:
a halogenated volatile anesthetic in an amount not greater than approximately 24% v/v of said formulation;
an emulsification adjuvant in an amount from approximately 8% to approximately 32% v/v of said formulation;
lecithin in an amount from approximately 1.2% to approximately 2.4% w/v of said formulation; and
a co-emulsifier in an amount not greater than approximately 1% w/v of said formulation.

2. An injectable anesthetic formulation in accordance with claim 1, wherein said halogenated anesthetic is selected from a group consisting of desflurane, isoflurane, enflurane, halothane, and sevoflurane.

3. An injectable anesthetic formulation in accordance with claim 1, wherein said halogenated anesthetic is isoflurane.

4. An injectable anesthetic formulation in accordance with claim 1, wherein said co-emulsifier is a polyoxypropylene/polyoxyethylene block copolymer.

5. An injectable anesthetic formulation in accordance with claim 4, wherein said co-emulsifier is poloxamer 188.

6. An injectable anesthetic formulation in accordance with claim 4, wherein said co-emulsifier has a formula:
HO(C₂H₄O)_{b}(C₃H₆O)ₐ(C₂H₄O)_{b}H
wherein a is an integer such that molecular weight represented by a polyoxypropylene portion of said copolymer is between approximately 900 to 15000, and b is an integer such that a molecular weight represented by a polyoxyethylene portion of said copolymer constitutes between approximately 5% and 90% of said copolymer.

7. An injectable anesthetic formulation in accordance with claim 1, wherein said emulsification adjuvant is soybean oil.

8. An injectable anesthetic formulation in accordance with claim 1, wherein said formulation further comprises glycerol in an amount of between approximately 1% to approximately 4% w/v of said formulation.

9. An injectable anesthetic formulation in accordance with claim 1, wherein said formulation further comprises water.

10. An injectable anesthetic formulation in accordance with claim 1, wherein said formulation further comprises a pH adjustor.

11. An injectable anesthetic formulation in accordance with claim 10, wherein said pH adjustor is sodium hydroxide.

12. An injectable anesthetic formulation comprising:
a halogenated anesthetic in an amount not greater than approximately 24% v/v of said formulation;
an emulsification adjuvant in an amount from approximately 8% to approximately 32% v/v of said formulation;
lecithin in an amount from approximately 1.2% to approximately 2.4% w/v of said formulation; and
a co-emulsifier in an amount not greater than approximately 1% w/v of said formulation;
a quantity of glycerol; and
a quantity of a pH adjustor sufficient to adjust a pH of said formulation to between approximately 6 and approximately 9.

13. An injectable anesthetic formulation in accordance with claim 12, wherein said halogenated anesthetic is selected from a group consisting of isoflurane, enflurane, halothane, and sevoflurane.

14. An injectable anesthetic formulation in accordance with claim 12, wherein said halogenated anesthetic is isoflurane.

15. An injectable anesthetic formulation in accordance with claim 1, wherein said co-emulsifier is a polyoxypropylene/polyoxyethylene block copolymer.

16. An injectable anesthetic formulation in accordance with claim 15, wherein said co-emulsifier is poloxamer 188.

17. An injectable anesthetic formulation in accordance with claim 15, wherein said co-emulsifier has a formula:
HO(C₂H₄O)_{b}(C₃H₆O)ₐ(C₂H₄O)_{b}H
wherein a is an integer such that molecular weight represented by a polyoxypropylene portion of said copolymer is between approximately 900 to 15000, and b is an integer such that a molecular weight represented by a polyoxyethylene portion of said copolymer constitutes between approximately 5% and 90% of said copolymer.

18. An injectable anesthetic formulation in accordance with claim 12, wherein said emulsification adjuvant is soybean oil.

19. An injectable anesthetic formulation in accordance with claim 12, wherein glycerol is present in an amount of between approximately 1% to approximately 4% w/v of said formulation.

20. An injectable anesthetic formulation in accordance with claim 1, wherein said formulation further comprises water in an amount sufficient for injection of said formulation

21. An injectable anesthetic formulation in accordance with claim 12, wherein said pH adjustor is sodium hydroxide.
